(19) United States
(12) Patent Application Publication
(10) Pub. No.: US 2023/0390066 A1
(43) Pub. Date: Dec. 7, 2023

(54) INFLATABLE PENILE PROSTHESIS HAVING A PLURALITY OF PUMP BULBS

(71) Applicant: Boston Scientific Scimed, Inc., Maple Grove, MN (US)

(72) Inventors: James Ryan Mujwid, Hudson, WI (US); Abigail Rae Brooks, Saint Louis Park, MN (US); John Anders Bostrom, Minneapolis, MN (US)

(21) Appl. No.: 18/325,518

(22) Filed: May 30, 2023

Related U.S. Application Data
(60) Provisional application No. 63/365,632, filed on Jun. 1, 2022.

(51) Int. Cl. A61F 2/26

(52) U.S. Cl. CPC A61F 2/26; A61F 2250/0013

(57) ABSTRACT

According to an aspect, an implant includes an inflatable member and a pump assembly. The pump assembly has a first pump bulb defining a first cavity, a second pump bulb defining a second cavity, a valve, and a valve housing. The pump assembly is configured to facilitate a transfer of fluid from the first cavity to the inflatable member in response to the first pump bulb being activated. The pump assembly is configured to facilitate a transfer of fluid from the second cavity to the inflatable member in response to the second pump bulb being activated.

### CROSS-REFERENCE TO RELATED APPLICATION

This application claims priority to U.S. Provisional Patent Application No. 63/365,632, filed on Jun. 1, 2022, entitled “INFLATABLE PENILE PROSTHESIS HAVING A PLURALITY OF PUMP BULBS”, the disclosure of which is incorporated by reference herein in its entirety.

### TECHNICAL FIELD

This disclosure relates generally to bodily implants and more specifically to bodily implants, such as penile prostheses that include a pump.

### BACKGROUND

One treatment for male erectile dysfunction is the implantation of a penile prosthesis that mechanically erects the penis. Additionally, some phalloplasty patients may receive a penile prosthesis. Some existing penile prostheses include inflatable cylinders or members that can be inflated or deflated using a pump mechanism. In some existing devices, the inflatable cylinder or member requires a relatively large amount of force to inflate. Additionally, in some existing devices, the pump mechanism may include a pump bulb that requires many sequential squeezes or activations to inflate the cylinder or member.

### SUMMARY

According to an aspect, an implant includes an inflatable member and a pump assembly. The pump assembly has a first pump bulb defining a first cavity, a second pump bulb defining a second cavity, a valve, and a valve housing. The pump assembly is configured to facilitate a transfer of fluid from the first cavity to the inflatable member in response to the first pump bulb being activated. The pump assembly is configured to facilitate a transfer of fluid from the second cavity to the inflatable member in response to the second pump bulb being activated.

In some embodiments, the valve housing is configured to be deformed to allow fluid to move from the inflatable member to the first cavity.

In some embodiments, the pump assembly includes a conduit extending between the valve housing and the first pump bulb. In some embodiments, the pump assembly includes a first conduit extending between the valve housing and the first pump bulb and a second conduit extending between the valve housing and the second pump bulb.

In some embodiments, valve is a first valve and is disposed within the valve housing, the pump assembly includes a second valve disposed within the valve housing. In some embodiments, the valve is a first valve and is disposed within the valve housing, the pump assembly includes a second valve disposed within the valve housing, a first biasing member configured to engage the first valve, and a second biasing member configured to engage the second valve. In some embodiments, the valve is a first valve and is disposed within the valve housing, the pump assembly includes a second valve disposed within the valve housing, the first valve having a spherical shape, the second valve having a first portion, a second portion, and a middle portion being disposed between the first portion and the second portion, the first portion having a first width, the second portion having a second width, the middle portion having a third width, the third width being smaller than the first width, the third width being smaller than the second width.

In some embodiments, the valve housing defines a first compartment and a second compartment, the valve being disposed within the first compartment, the first compartment having a first portion, a second portion, and a third portion disposed between the first portion and the second portion, the first portion having a first width, the second portion having a second width, the third portion having a third width, the third width being smaller than the first width, the third width being smaller than the second width. In some embodiments, the valve is a first valve, the pump assembly includes a second valve, the valve housing defines a first compartment and a second compartment, the first valve being disposed within the first compartment, the second valve being disposed within the second compartment, the second compartment having a first portion, a second portion, and a third portion disposed between the first portion and the second portion, the first portion having a first width, the second portion having a second width, the third portion having a third width, the third width being smaller than the first width, the third width being smaller than the second width.

In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width. In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a portion of the valve housing to form a seal.

In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal. In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal, the seat surface being configured to be moved from the seat portion of the valve housing when the valve housing is deformed.

In some embodiments, the valve housing is configured to be deformed to allow fluid to move from the inflatable member to the first cavity, the valve housing is configured to be deformed when the valve housing is compressed. In some embodiments, the valve housing is configured to be deformed to allow fluid to move from the inflatable member to the first cavity, the valve housing is configured to be deformed when the valve housing is compressed by a user.

According to another aspect, a method includes squeezing a first pump bulb of a bodily implant to transfer fluid from a cavity of the first pump bulb to an inflatable member of the bodily implant; and squeezing a second pump bulb of the bodily implant to transfer fluid from a cavity of the second pump bulb to the inflatable member.

In some embodiments, the method includes deflating the inflatable member, the deflating including deforming a valve housing of the bodily implant to unseat a valve of the bodily implant.

In some embodiments, the method includes deflating the inflatable member, the deflating including deforming a valve housing of the bodily implant to unseat a valve of the bodily implant, the deflating includes squeezing a portion of the valve housing.

In some embodiments, the squeezing the first pump bulb includes squeezing the first pump bulb a single time.

In some embodiments, the squeezing the first pump bulb includes squeezing the first pump bulb a single time, the squeezing the second pump bulb includes squeezing the second pump bulb a single time.

### BRIEF DESCRIPTION OF THE DRAWINGS

FIG. 1 schematically illustrates a penile prosthesis according to an embodiment.

FIG. 2 illustrates a penile prosthesis according to another embodiment.

FIG. 3 illustrates a portion of the penile prothesis of FIG. 2.

FIGS. 4 and 5 illustrate a portion of a penile prothesis according to another embodiment.

FIG. 6 illustrates a portion of a penile prothesis according to another embodiment.

FIG. 7 illustrates a penile prosthesis according to another embodiment.

FIG. 8 is a flow chart of a method according to an embodiment.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as penile prostheses or other bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The embodiments discussed herein may improve the performance of an inflatable member of the device. For example, the inflatable member may have improved stiffness or rigidity, improved reliability, or improved deflation or inflation times. In some embodiments, inflating the inflatable member may be facilitated by requiring less force, less pressure, or less fluid transfer to inflate the inflatable member. Additionally, in some embodiments, the inflatable member may be inflated with a single squeeze of each pump bulb.

The embodiments may include an inflatable penile prosthesis having a pump assembly and an inflatable member. The inflatable member may be implanted into the corpus cavernosae of a patient or user and the pump assembly may be implanted in the scrotum.

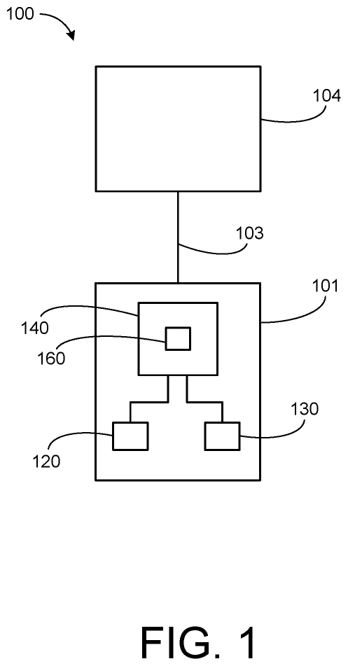

FIG. 1 schematically illustrates an inflatable penile prosthesis 100 according to an aspect. The inflatable penile prosthesis 100 includes a cylinder or inflatable member 104 and a pump assembly 101 configured to transfer fluid to and from the inflatable member 104. In some examples, the inflatable member 104 may be implanted into the corpus cavernosae of the user and the pump assembly 101 may be implanted in the scrotum of the user.

The inflatable member 104 may be capable of expanding upon the injection of fluid into a cavity of the inflatable member 104. For instance, upon injection of the fluid into the inflatable member 104, the inflatable member 104 may increase its length and/or width, as well as increase its rigidity. In some examples, the inflatable member 104 may include a pair of cylinders or at least two cylinders, e.g., a first cylinder member and a second cylinder member. The volumetric capacity of the inflatable member 104 may depend on the size of the cylinders. In some examples, the volume of fluid in each cylinder may vary from about 10 milliliters in smaller cylinders and to about milliliters in larger sizes. In some examples, the first cylinder member may be larger than the second cylinder member. In other examples, the first cylinder member may be the same size as the second cylinder member.

The inflatable penile prosthesis 100 includes a conduit connector 103. The conduit connector 103 may define a lumen configured to transfer the fluid to and from the pump assembly 101. The conduit connector 103 may be coupled to the pump assembly 101 and to the inflatable member 104. The conduit connector 103 may include a single or multiple tube members for transferring the fluid between the pump assembly 101 and the inflatable member 104.

In the illustrated embodiment, the pump assembly 101 includes a first pump bulb 120, a second pump bulb 130, and a valve housing 140. The first pump bulb 120 and the second pump bulb 130 are operatively coupled to the valve housing 140. For example, in some embodiments, the first pump bulb 120 and the second pump bulb 130 are fluidically coupled to the valve housing 140. While the illustrated embodiment, includes a first and a second pump bulb, in other embodiments, the pump assembly includes only one pump bulb.

The first pump bulb 120 may include a cavity or a cavity portion. The cavity or cavity portion may be configured to retain fluid that is configured to be transferred to the inflatable member 104 to place the inflatable member in an inflated configuration. In some embodiments, a single squeeze of the first pump bulb 120 by the user will cause the fluid disposed within the cavity or cavity portion of the first pump bulb to be transferred to the inflatable member 104.

Similarly, the second pump bulb 130, may include a cavity or a cavity portion. The cavity or cavity portion may be configured to retain fluid that is configured to be transferred to the inflatable member 104 to place the inflatable member in an inflated configuration. In some embodiments, a single squeeze of the second pump bulb 130 by the user will cause the fluid disposed within the cavity or cavity portion of the second pump bulb to be transferred to the inflatable member 104.

The pump assembly 101 also includes a valve housing 140 and a valve 160. The valve housing 140 is operatively coupled to the first pump bulb 120. In the illustrated embodiment, the valve housing 140 is fluidically coupled to the pump bulbs. The valve 160 is configured to regulate the flow of fluid between the inflatable member 104 and the first pump bulb 120.

In some embodiments, the valve housing 140 is configured to be deformed to allow the fluid to flow from the inflatable member 104 to the pump bulbs. For example, in some embodiments, the valve housing 140 is formed of a material that may be deformed upon a compression force. In some embodiments, a user may squeeze or otherwise compress the valve housing 140 to deform the valve housing 140. In some embodiments, the deformation of the valve housing 140 causes the valve 160 to become unseated and allow the fluid in the inflatable member 104 to flow to the pump bulbs to place the inflatable member 104 in a deflated configuration.

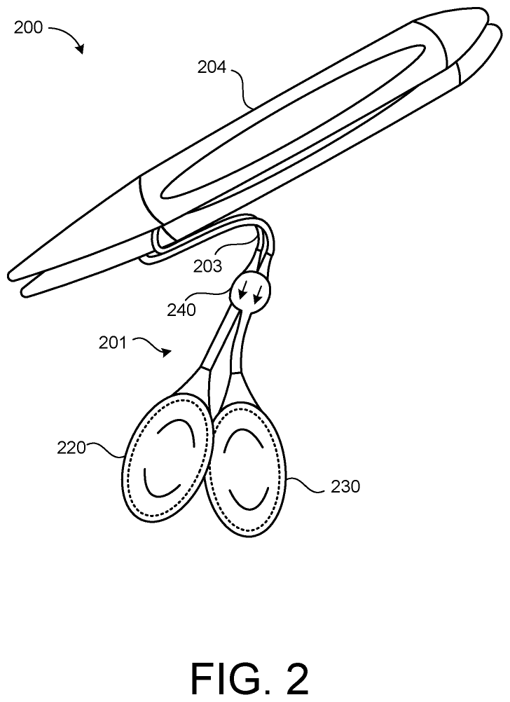

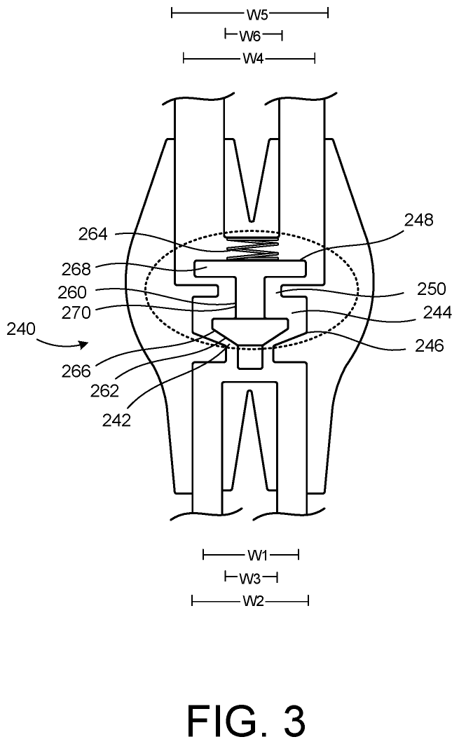

FIG. 2 illustrates a penile prosthesis 200 according to an aspect. FIG. 3 illustrates a pump assembly 201 of the penile prosthesis 200.

The inflatable penile prosthesis 200 includes an inflatable member 204 and a pump assembly 201 configured to transfer fluid to and from the inflatable member 204. In the illustrated embodiment, the inflatable member 204 includes two or a pair of inflatable cylinders. In other embodiments, the inflatable member may include a single inflatable cylinder.

The inflatable member 204 may be implanted into the corpus cavernosae of the user or patient and the pump assembly 201 may be implanted in the scrotum of the user or patient.

The inflatable member 204 may be capable of expanding upon the injection of fluid into a cavity of the inflatable member 204. For instance, upon injection of the fluid into the inflatable member 204, the inflatable member 204 may increase its length and/or width, as well as increase its rigidity. In some examples, the inflatable member 204 may include a pair of cylinders or at least two cylinders, e.g., a first cylinder member and a second cylinder member. The volumetric capacity of the inflatable member 204 may depend on the size of the cylinders. In some examples, the volume of fluid in each cylinder may vary from about 10 milliliters in smaller cylinders and to about milliliters in larger sizes. In some examples, one cylinder member may be larger than the other cylinder member. In other examples, the first cylinder member may be the same size as the second cylinder member.

The inflatable penile prosthesis 200 includes a conduit connector 203. The conduit connector 203 defines a lumen configured to transfer the fluid to and from the pump assembly 201 and the inflatable member 204. The conduit connector 203 is coupled to the pump assembly 201 and to the inflatable member 204. The conduit connector 203 may include a single or multiple tube members for transferring the fluid between the pump assembly 201 and the inflatable member 204.

In the illustrated embodiment, the pump assembly 201 includes a first pump bulb 220, a second pump bulb 230, and a valve housing 240. The first pump bulb 220 and the second pump bulb 230 separate from each other and are each operatively coupled to the valve housing 240. For example, in some embodiments, the first pump bulb 220 and the second pump bulb 230 are fluidically coupled to the valve housing 240.

The first pump bulb 220 may include or define a cavity or a cavity portion. The cavity or cavity portion may be configured to retain fluid that is configured to be transferred to the inflatable member 204 to place the inflatable member in an inflated configuration. In some embodiments, a single squeeze of the first pump bulb 220 by the user will cause the fluid disposed within the cavity or cavity portion of the first pump bulb to be transferred to the inflatable member 204.

Similarly, the second pump bulb 230, may include or define a cavity or a cavity portion. The cavity or cavity portion may be configured to retain fluid that is configured to be transferred to the inflatable member 204 to place the inflatable member in an inflated configuration. In some embodiments, a single squeeze of the second pump bulb 230 by the user will cause the fluid disposed within the cavity or cavity portion of the second pump bulb to be transferred to the inflatable member 204.

The pump assembly 201 includes a valve housing 240 and a valve 260. The valve housing 240 is operatively coupled to the first pump bulb 220 and to the second pump bulb 230. In the illustrated embodiment, the valve housing 240 is fluidically coupled to the first pump bulb 220 and to the second pump bulb 230. The valve 260 is configured to regulate the flow of fluid between the inflatable member 204 and the first pump bulb 220 and the second pump bulb 230.

The valve 260 includes a seat portion or surface 262 that is configured to engage a seat or sealing portion 242 of the valve housing 240. In the illustrated embodiment, the valve 260 is biased towards the seat or sealing portion 242 of the valve housing 240. Specifically, in the illustrated embodiment, a spring or other biasing member 264 is disposed such that it biases the seat or sealing portion 262 of the valve 260 towards the seat or sealing portion 242 of the valve housing 240.

In the illustrated embodiment, the valve 260 includes a first end portion 266, a second end portion 268, and a middle portion 270 disposed between the first end portion 266 and the second end portion 268. The first end portion 266 has a width W1. The second end portion 268 has a width W2. The middle portion 270 has a width W3. The width W3 of the middle portion 270 is smaller than the width W1 of the first end portion 266. The width W3 of the middle portion 270 is smaller than the width W2 of the second end portion 268.

The valve housing 240 defines a cavity 244. The valve 260 is disposed within the cavity 244. The cavity 244 includes a first portion 246, a second portion 248, and a middle portion 250. The first portion 246 of the cavity 244 has a width W4. The second portion 248 of the cavity 244 has a width W5. The middle portion 250 has a width W6. The width W6 of the middle portion 250 is smaller than the width W4 of the first portion 246. The width W6 of the middle portion 250 is smaller than the width W5 of the second portion 248.

The first end portion 266 of the valve 260 is disposed within the first portion 246 of the cavity 244. The second end portion 268 of the valve 260 is disposed within the second portion 248 of the cavity 244. The middle portion 270 of the valve 260 is disposed within the middle portion 250 of the cavity 244.

The valve housing 240 is configured to be deformed to allow the fluid to flow from the inflatable member 204 to the first pump bulb 220. For example, in some embodiments, the valve housing 240 is formed of a material that may be deformed upon a compression force. In some embodiments, a user may squeeze or otherwise compress the valve housing 240 to deform the valve housing 240. In some embodiments, the deformation of the valve housing 240 causes the valve 260 to become unseated and allow the fluid in the inflatable member 204 to flow to the first pump bulb 220 to place the inflatable member 204 in a deflated configuration. In some embodiments, the deformation of the valve housing 240 causes the valve 260 to become unseated and allow the fluid in the inflatable member 204 to flow to the first pump bulb 220 and to the second pump bulb 230 to place the inflatable member 204 in a deflated configuration. In some embodiments, the valve housing 240 includes an outer surface that includes finger pads to facilitate the user squeezing or compressing the valve housing 240.

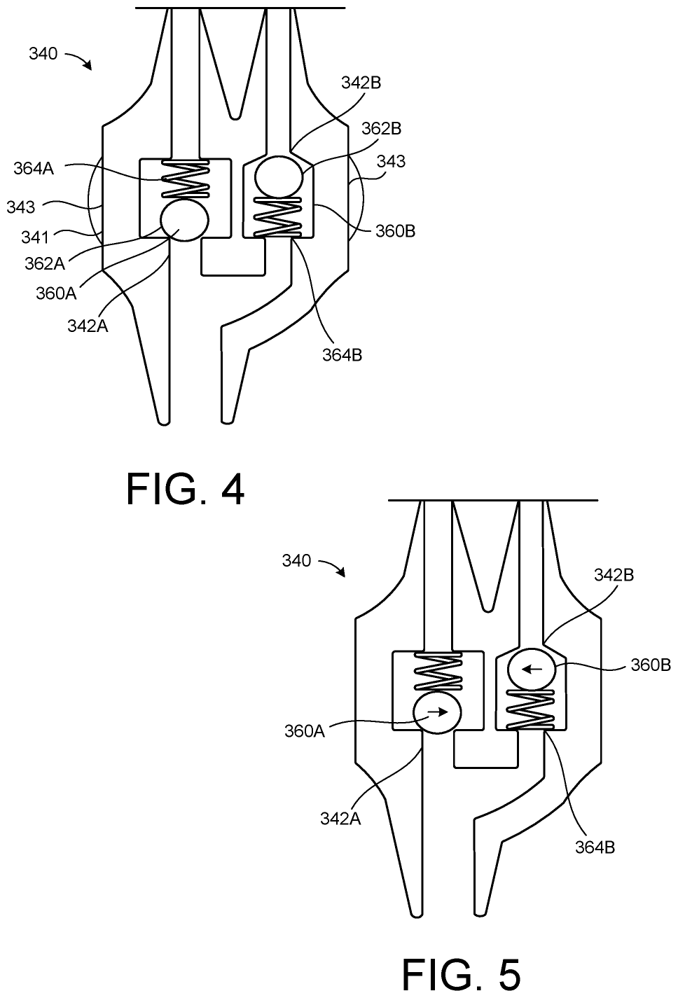

FIGS. 4 and 5 illustrate a valve housing 340 of a penile prothesis according to another embodiment. The valve housing 340 may be operatively coupled to a first pump bulb and to the second pump bulb. For example, the valve housing 340 may be fluidically coupled to the first pump bulb and to the second pump bulb. In the illustrated embodiment, the valve housing 340 includes a first valve 360A and a second valve 360B. The valves 360A and 360B are configured to regulate the flow of fluid between the inflatable member and the first pump bulb and the second pump bulb.

The valve 360A includes a seat portion or surface 362A that is configured to engage a seat or sealing portion 342A of the valve housing 340. In the illustrated embodiment, the valve 360A is biased towards the seat or sealing portion 342A of the valve housing 340. Specifically, in the illustrated embodiment, a spring or other biasing member 364A is disposed such that it biases the seat or sealing portion 362A of the valve 360A towards the seat or sealing portion 342A of the valve housing 340.

The valve 360B includes a seat portion or surface 362B that is configured to engage a seat or sealing portion 342B of the valve housing 340. In the illustrated embodiment, the valve 360B is biased towards the seat or sealing portion 342B of the valve housing 340. Specifically, in the illustrated embodiment, a spring or other biasing member 364B is disposed such that it biases the seat or sealing portion 362B of the valve 360B towards the seat or sealing portion 342B of the valve housing 340.

The valve housing 340 is configured to be deformed to allow the fluid to flow from the inflatable member to the pump bulbs. For example, in some embodiments, the valve housing 340 is formed of a material that may be deformed upon a compression force. In some embodiments, a user may squeeze or otherwise compress the valve housing 340 to deform the valve housing 340. In some embodiments, the deformation of the valve housing 340 causes the valves 360A and 360B to become unseated and allow the fluid in the inflatable member to flow to the pump bulbs to place the inflatable member in a deflated configuration. For example, in the illustrated embodiment, the valves 360A and 360B to move towards each other (as shown with the arrows in FIG. 5) such that they become unseated.

In the illustrated embodiment, the valve housing 340 includes an outer surface 341. The outer surface 341 includes or defines finger pads 343 to facilitate the user squeezing or compressing the valve housing 340.

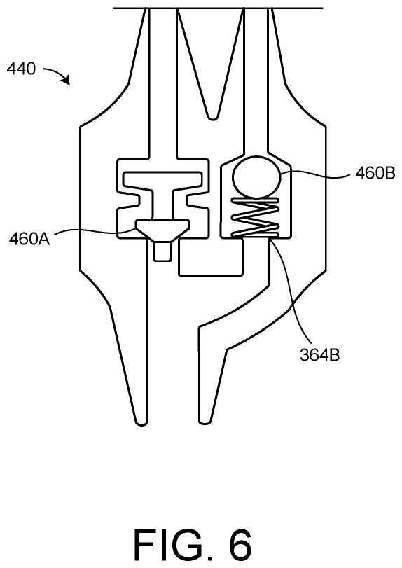

In the illustrated embodiment, the valves 360A and 360B are spheres or spherical in shape. In other embodiments, the valves have different shapes. As best illustrated in FIG. 6, in one embodiment, a valve housing 440 includes a first valve 460A and a second valve 460B. The valve 460A has a first shape and the valve 460B has a second shape. The first shape is different than the second shape.

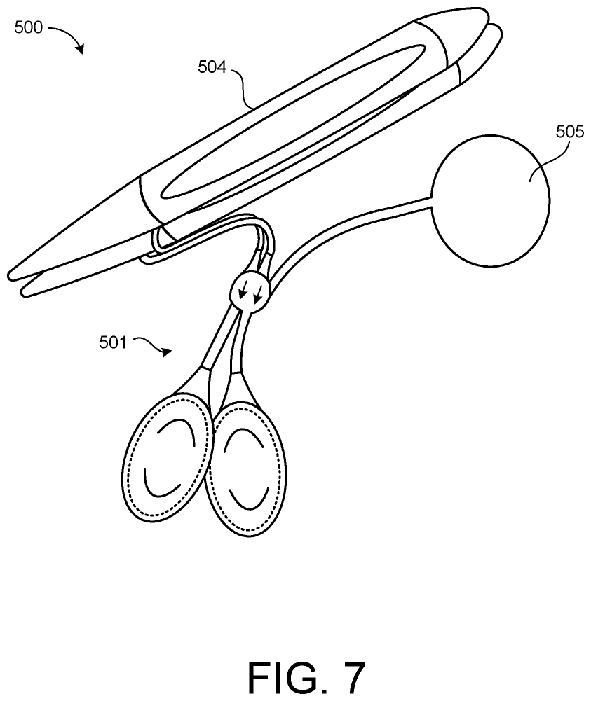

FIG. 7 illustrates a penile prosthesis 500 according to another embodiment. The penile prosthesis 500 includes a cylinder or inflatable member 504, a pump assembly 501, and a reservoir 505. The pump assembly 501 is configured to transfer fluid to and from the inflatable member 504. The reservoir 505 may define a cavity or container and may be configured to retain fluid. In some embodiments, the inflatable member 504 may be implanted into the corpus cavernosae of the user, the pump assembly 501 may be implanted in the scrotum of the user, and the reservoir 505 may be implanted into an abdomen or other portion of the body of the user.

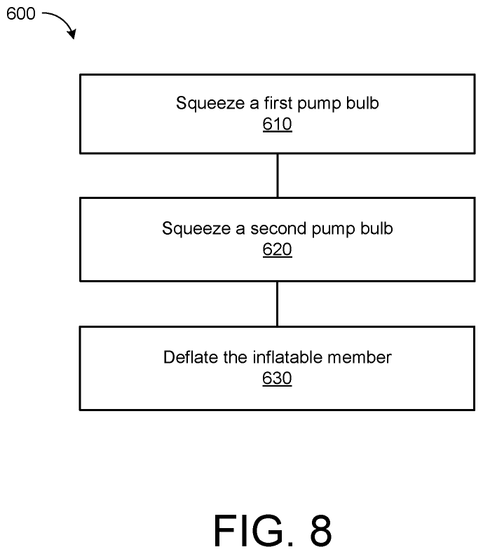

FIG. 8 is a flow chart of a method 600 according to an embodiment. At 610, a first pump bulb is squeezed to inject or transfer fluid to an inflatable member. In some embodiments, the transfer of fluid is from the first pump bulb to the inflatable member and such transfer of fluid at least partially inflates the inflatable member. At 620, a second pump bulb is squeezed to inject or transfer fluid to the inflatable member. In some embodiments, the transfer of fluid is from the second pump bulb to the inflatable member. In some embodiments, the injection of the fluid from the second pump bulb places the inflatable member in its inflated configuration.

At 630, the inflatable member is deflated. In some embodiments, a valve housing is deformed to deflate the inflatable member. In some embodiments, the deformation of the valve housing allows fluid in the inflatable member to move from the inflatable member, past a valve member, and into the cavities of the pump bulbs. In some embodiments, the deformation of the valve housing causes the valve to move from a seated or sealed position.

In other embodiments, a user may fold the inflatable member such as fold the inflatable member in half to deflate the inflatable member. In some embodiments, the pressure of the folding of the inflatable member causes the valve housing to deform and allow fluid to pass to the cavities of the pump bulbs. In other embodiments, the valve housing may define slots or grooves that allow the fluid to pass the valve when the pressure within the inflatable member is increased, such as by folding or bending the inflatable member.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implant, comprising:
an inflatable member; and
a pump assembly having a first pump bulb defining a first cavity, a second pump bulb defining a second cavity, a valve, and a valve housing, the pump assembly being configured to facilitate a transfer of fluid from the first cavity to the inflatable member in response to the first pump bulb being activated, the pump assembly being configured to facilitate a transfer of fluid from the second cavity to the inflatable member in response to the second pump bulb being activated.

2. The implant of claim 1, wherein the valve housing is configured to be deformed to allow fluid to move from the inflatable member to the first cavity.

3. The implant of claim 1, wherein the pump assembly includes a conduit extending between the valve housing and the first pump bulb.

4. The implant of claim 1, wherein the pump assembly includes a first conduit extending between the valve housing and the first pump bulb and a second conduit extending between the valve housing and the second pump bulb.

5. The implant of claim 1, wherein the valve is a first valve and is disposed within the valve housing, the pump assembly includes a second valve disposed within the valve housing.

6. The implant of claim 1, wherein the valve is a first valve and is disposed within the valve housing, the pump assembly includes a second valve disposed within the valve housing, a first biasing member configured to engage the first valve, and a second biasing member configured to engage the second valve.

7. The implant of claim 1, wherein the valve is a first valve and is disposed within the valve housing, the pump assembly includes a second valve disposed within the valve housing, the first valve having a spherical shape, the second valve having a first portion, a second portion, and a middle portion being disposed between the first portion and the second portion, the first portion having a first width, the second portion having a second width, the middle portion having a third width, the third width being smaller than the first width, the third width being smaller than the second width.

8. The implant of claim 1, wherein the valve housing defines a first compartment and a second compartment, the valve being disposed within the first compartment, the first compartment having a first portion, a second portion, and a third portion disposed between the first portion and the second portion, the first portion having a first width, the second portion having a second width, the third portion having a third width, the third width being smaller than the first width, the third width being smaller than the second width.

9. The implant of claim 1, wherein the valve is a first valve, the pump assembly includes a second valve, the valve housing defines a first compartment and a second compartment, the first valve being disposed within the first compartment, the second valve being disposed within the second compartment, the second compartment having a first portion, a second portion, and a third portion disposed between the first portion and the second portion, the first portion having a first width, the second portion having a second width, the third portion having a third width, the third width being smaller than the first width, the third width being smaller than the second width.

10. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width.

11. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a portion of the valve housing to form a seal.

12. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal.

13. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal, the seat surface being configured to be moved from the seat portion of the valve housing when the valve housing is deformed.

14. The implant of claim 1, wherein the valve housing is configured to be deformed to allow fluid to move from the inflatable member to the first cavity, the valve housing is configured to be deformed when the valve housing is compressed.

15. The implant of claim 1, wherein the valve housing is configured to be deformed to allow fluid to move from the inflatable member to the first cavity, the valve housing is configured to be deformed when the valve housing is compressed by a user.

16. A method, comprising:
squeezing a first pump bulb of a bodily implant to transfer fluid from a cavity of the first pump bulb to an inflatable member of the bodily implant; and
squeezing a second pump bulb of the bodily implant to transfer fluid from a cavity of the second pump bulb to the inflatable member.

17. The method of claim 16, further comprising:
deflating the inflatable member, the deflating including deforming a valve housing of the bodily implant to unseat a valve of the bodily implant.

18. The method of claim 16, further comprising:
deflating the inflatable member, the deflating including deforming a valve housing of the bodily implant to unseat a valve of the bodily implant, the deflating includes squeezing a portion of the valve housing.

19. The method of claim 16, wherein the squeezing the first pump bulb includes squeezing the first pump bulb a single time.

20. The method of claim 16, wherein the squeezing the first pump bulb includes squeezing the first pump bulb a single time, the squeezing the second pump bulb includes squeezing the second pump bulb a single time.
